(19) United States
(12) Patent Application Publication
(10) Pub. No.: US 2012/0172693 A1
(43) Pub. Date: Jul. 5, 2012

(54) Pump Arrangement in Continuous Analyte Monitoring

(76) Inventors: Varun Boriah, Union City, CA (US); Janet Tamada, Stanford, CA (US); Arvind N. Jina, San Jose, CA (US); Michael J. Tierney, San Jose, CA (US); Shashi P. Desai, San Jose, CA (US)

(21) Appl. No.: 12/985,234

(22) Filed: Jan. 5, 2011

(51) Int. Cl. A61B 5/145

(52) U.S. Cl. 600/365

(57) ABSTRACT

A method of monitoring an analyte (such as, e.g., glucose) including the following steps: diffusing the analyte from a sampling location into a sensing fluid within a sensing chamber; detecting a concentration of the analyte in the sensing fluid; moving flushing fluid into the sensing chamber and simultaneously removing sensing fluid from the sensing chamber; permitting the flushing fluid to remain in the sensing chamber without flowing for a dwell time; removing the flushing fluid from the sensing chamber after the dwell time expires; and, after removing the flushing fluid from the sensing chamber, moving sensing fluid into the sensing chamber. The invention also includes an analyte monitoring device performing this method.

### INCORPORATION BY REFERENCE

All publications and patent applications mentioned in this specification are herein incorporated by reference to the same extent as if each individual publication or patent application was specifically and individually indicated to be incorporated by reference.

### BACKGROUND OF THE INVENTION

The invention relates to methods and apparatus for monitoring the presence and/or concentration of an analyte or analytes, such as for monitoring the glucose level of a person having diabetes. More specifically, the invention relates to systems, devices, sensors and tools and methods associated therewith for monitoring analyte levels continuously, or substantially continuously.

Diabetes is a chronic, life-threatening disease for which there is no known cure at present. It is a syndrome characterized by hyperglycemia and relative insulin deficiency. Diabetes affects more than 120 million people worldwide, and is projected to affect more than 220 million people by the year 2020. There are 20.8 million children and adults in the United States, or 7% of the population, who have diabetes. Of these people, 14.6 million have been diagnosed with the disease, while unfortunately nearly one-third remain undiagnosed. It is estimated that one out of every three children today will develop diabetes sometime during their lifetime. Diabetes is usually irreversible, and can lead to a variety of severe health complications, including coronary artery disease, peripheral vascular disease, blindness and stroke. The Center for Disease Control (CDC) has reported that there is a strong association between being overweight, obesity, diabetes, high blood pressure, high cholesterol, asthma and arthritis. Individuals with a body mass index of 40 or higher are more than 7 times more likely to be diagnosed with diabetes.

There are two main types of diabetes, Type I diabetes (insulin-dependent diabetes mellitus) and Type II diabetes (non-insulin-dependent diabetes mellitus). Varying degrees of insulin secretory failure may be present in both forms of diabetes. In some instances, diabetes is also characterized by insulin resistance. Insulin is the key hormone used in the storage and release of energy from food.

As food is digested, carbohydrates are converted to glucose and glucose is absorbed into the blood stream primarily in the intestines. Excess glucose in the blood, e.g. following a meal, stimulates insulin secretion, which promotes entry of glucose into the cells, which controls the rate of metabolism of most carbohydrates.

Insulin secretion functions to control the level of blood glucose both during fasting and after a meal, to keep the glucose levels at an optimum level. In a non-diabetic person blood glucose levels are typically between 80 and 90 mg/dL of blood during fasting and between 120 to 140 mg/dL during the first hour or so following a meal. For a person with diabetes, the insulin response does not function properly (either due to inadequate levels of insulin production or insulin resistance), resulting in blood glucose levels below 80 mg/dL during fasting and well above 140 mg/dL after a meal.

Currently, persons suffering from diabetes have limited options for treatment, including taking insulin orally or by injection. In some instances, controlling weight and diet can impact the amount of insulin required, particularly for non-insulin dependent diabetics. Monitoring blood glucose levels is an important process that is used to help diabetics maintain blood glucose levels as near as normal as possible throughout the day.

The blood glucose self-monitoring market is the largest self-test market for medical diagnostic products in the world, with a size of approximately over $3 billion in the United States and $7.0 billion worldwide. It is estimated that the worldwide blood glucose self-monitoring market will amount to $9.0 billion by 2008. Failure to manage the disease properly has dire consequences for diabetics. The direct and indirect costs of diabetes exceed $130 billion annually in the United States—about 20% of all healthcare costs.

There are two main types of blood glucose monitoring systems used by patients: non-continuous systems, also known as single point, discrete or episodic, and continuous systems. Episodic systems consist of meters and tests strips and require blood samples to be drawn from fingertips or alternate sites, such as forearms and legs (e.g. OneTouch® Ultra by LifeScan, Inc., Milpitas, Calif., a Johnson & Johnson company). These systems rely on lancing and manipulation of the fingers or alternate blood draw sites, which can be extremely painful and inconvenient, particularly for children.

Continuous monitoring sensors are generally implanted subcutaneously and measure glucose levels in the interstitial fluid at various periods throughout the day, providing data that shows trends in glucose measurements over a short period of time. These sensors are painful during insertion and usually require the assistance of a health care professional. Further, these sensors are intended for use during only a short duration (e.g., monitoring for a matter of days to determine a blood sugar pattern). Subcutaneously implanted sensors also frequently lead to infection and immune response complications. Another major drawback of currently available continuous monitoring devices is that they require frequent, often daily, calibration using blood glucose results that must be obtained from painful finger-sticks using traditional meters and test strips. This calibration, and re-calibration, is required to maintain sensor accuracy and sensitivity, but it can be cumbersome and inconvenient.

Data from various studies such as the Diabetes Control and Complications trial (DCCT) show that frequent testing of blood glucose levels is essential to improve the quality of life for diabetics. However, most diabetics avoid frequent testing because of the inconvenience, fear, and pain of pricking their fingers or alternate sites to obtain blood samples. Thus there is a need to develop simple glucose monitoring systems that eliminate or minimize these barriers to frequent testing. With some embodiments of the proposed present invention a user or diabetic patient can obtain 20 or more glucose test results over a two or three day period thus allowing frequent measurements on a daily basis.

US 2006/0219576 discloses an analyte monitor that permits glucose from a user's interstitial fluid to diffuse into fluid within a sensing channel and measures the concentration of the glucose in the fluid within the sensing channel using a glucose sensor. To calibrate this device, fluid of known glucose concentration is moved from a source reservoir into the sensing channel, thereby displacing the fluid that was already in the sensing channel and moving the displaced fluid into a waste reservoir. Similar devices are described in US 2008/0154107, US 2008/0234562 and US 2009/0131778. The disclosures of these published patent applications are incorporated herein by reference.

### SUMMARY OF THE INVENTION

The accuracy of analyte monitors relying on a periodic flushing of sensing fluid from a sensing area or chamber depends in part on the completeness of the sensing fluid replacement. Lingering quantities of analyte or interfering species can affect the accuracy of subsequent analyte diffusion and sensing. Optimization of the flushing operation is therefore an important aspect of the overall operation of the sensor.

One aspect of the invention provides a method of monitoring an analyte (such as, e.g., glucose) including the following steps: diffusing the analyte from a sampling location into a sensing fluid within a sensing chamber; detecting a concentration of the analyte in the sensing fluid; moving flushing fluid into the sensing chamber and simultaneously removing sensing fluid from the sensing chamber; permitting the flushing fluid to remain in the sensing chamber without flowing for a dwell time; removing the flushing fluid from the sensing chamber after the dwell time expires; and, after removing the flushing fluid from the sensing chamber, moving sensing fluid into the sensing chamber.

In some embodiments, the dwell time is between 1 second and 30 seconds. The steps of moving and removing the flushing fluid may be performed by a pulsatile pump.

In some embodiments, the sampling location is a subject's interstitial fluid. In some such embodiments, the diffusing step may include the step of diffusing the analyte from the interstitial fluid via fluid paths formed through a stratum corneum layer of the subject's skin. The method may also include the step of forming the fluid paths through the stratum corneum.

In some embodiments, the flushing fluid and the sensing fluid are the same. In some embodiments, the flushing fluid is an aqueous buffer electrolyte solution. In some embodiments, the flushing fluid is calibration fluid, and the method further includes the step of calibrating an analyte sensor prior to removing the flushing fluid from the sensing chamber. In some embodiments, the analyte diffuses from the sampling location into the sensing fluid without using a dialysis membrane.

Another aspect of the invention provides an analyte monitoring device having a sampling member; a sensing chamber in fluid communication with the sampling member; an analyte sensor in fluid contact with the sensing chamber; and a pump (such as, e.g., a pulsatile pump) programmed to move flushing fluid into the sensing chamber and remove sensing fluid from the sensing chamber so that the flushing fluid remains in the sensing chamber without flowing for a dwell time. In some embodiments, the sampling member has a fluid path and/or a tissue piercing element.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 is a cross-sectional view of one embodiment of an analyte monitor according to an embodiment of the invention.

FIG. 2 is an exploded view of an embodiment of an analyte monitor according to an embodiment of the invention.

FIG. 3 is a cross-sectional view of yet another embodiment of an analyte monitor according to an embodiment of the invention.

### DETAILED DESCRIPTION OF THE INVENTION

While many of the exemplary embodiments disclosed herein are described in relation to monitoring glucose levels in people with diabetes, it should be understood that aspects of the invention are useful in monitoring glucose levels in people without diabetes, or for monitoring an analyte or analytes other than glucose. For example, the present invention may be used in monitoring the concentration, or presence, of other analytes such as lactate, acetyl choline, amylase, bilirubin, cholesterol, chorionic gonadotropin, creatine kinase (e.g., CK-MB), creatine, DNA, fructosamine, glutamine, growth hormones, hematocrit, hemoglobin (e.g. HbAlc), hormones, ketones, lactate, oxygen, peroxide, prostate-specific antigen, prothrombin, RNA, thyroid stimulating hormone, troponin, drugs such as antibiotics (e.g., gentamicin, vancomycin), digitoxin, digoxin, drugs of abuse, theophylline, and warfarin. Accordingly, while the invention will be described in connection with glucose monitoring, it should be understood that the invention may be used to monitor other analytes as well.

The present invention provides a significant advance in biosensor and glucose monitoring technology: portable, virtually non-invasive, self-calibrating, integrated and non-implanted sensors which continuously indicate the user's blood glucose concentration, enabling swift corrective action to be taken by the patient. The sensor and monitor of this invention may be used to measure other analytes as well, such as electrolytes like sodium or potassium ions. As will be appreciated by persons of skill in the art, the glucose sensor can be any suitable sensor including, for example, an electrochemical sensor an optical sensor.

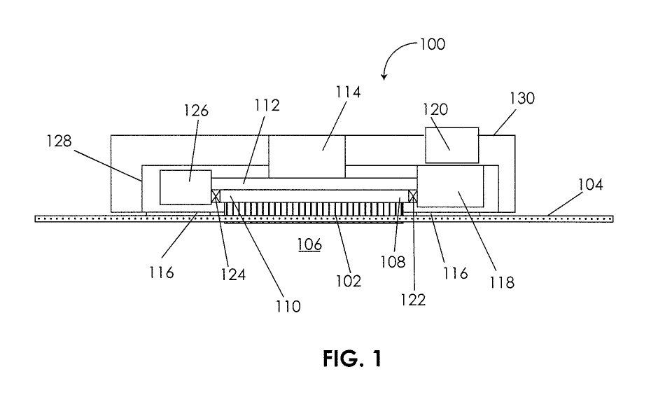

FIG. 1 shows a schematic cross-section of one embodiment of the invention in use. The glucose monitor 100 has an array of tissue piercing elements 102 forming and/or defining fluid paths through the stratum corneum 104 of a user into the interstitial fluid 106 beneath the stratum corneum. Suitable microneedle arrays include those described in Stoeber et al. U.S. Pat. No. 6,406,638; US Patent Appl. Publ. No. 2005/0171480; and US Patent Appl. Publ. No. 2006/0025717. The needles in array 102 are hollow and have open distal ends, and their interiors communicate with a sensing zone 110 within a sensor channel 108. Sensing zone 110 is therefore in fluid communication with interstitial fluid 106 through microneedle array 102. In this embodiment, sensing zone 110 and the tissue piercing elements or fluid paths 102 are pre-filled with sensing fluid prior to the first use of the device. Thus, when the device is applied to the user's skin and the tissue piercing elements or fluid paths pierce the stratum corneum of the skin, there is substantially no net fluid transfer from the interstitial fluid into the tissue piercing elements or fluid paths. Rather, glucose diffuses from the interstitial fluid into the sensing fluid within the needles, as described below.

Disposed above and in fluid communication with sensor channel 108 is a glucose sensor 112. In some embodiments, glucose sensor is an electrochemical glucose sensor that generates an electrical signal (current, voltage or charge) whose value depends on the concentration of glucose in the fluid within sensing zone 110. Details of suitable glucose sensors may be found, e.g., in US 2008/0234562 and US 2009/0131778.

Sensor electronics element 114 receives the voltage signal from sensor 112. In some embodiments, sensor electronics element 114 uses the sensed signal to compute a glucose concentration and display it. In other embodiments, sensor electronics element 114 transmits the sensed signal, or information derived from the sensed signal, to a remote device, such as through wireless communication. Glucose monitor 100 is held in place on the skin 104 by one or more adhesive pads 116.

In some embodiments, glucose monitor 100 has a built-in sensor calibration system. A reservoir 118 contains a sensing fluid having, e.g., a glucose concentration between about 0 and about 400 mg/dl. In some embodiments, the glucose concentration in the sensing fluid is selected to be below the glucose sensing range of the sensor. The sensing fluid may also contain buffers, preservatives or other components in addition to the glucose. Upon actuation of a pump 120 manually (e.g., via plunger or other actuator) or automatically, sensing fluid is forced from reservoir 118 through a check valve 122 (such as a flap valve) into sensing channel 108. Sensing fluid within channel 108 is displaced through a second check valve 124 (e.g., a flap valve) into a waste reservoir 126. Check valves or similar gating systems are used to prevent contamination.

Because the fresh sensing fluid has a known glucose concentration, sensor 112 can be calibrated at this value to set a base line. After calibration, the sensing fluid in channel 108 remains stationary, and glucose from the interstitial fluid 106 diffuses through tissue piercing elements or fluid paths 102 into the sensing zone 110. Changes in the glucose concentration from over time reflect differences between the calibration glucose concentration of the sensing fluid in the reservoir 118 and the glucose concentration of the interstitial fluid which can be correlated with the actual blood glucose concentration of the user using proprietary algorithms. Because of possible degradation of the sensor or loss of sensor sensitivity over time, the device may be periodically recalibrated by operating actuator 120 manually or automatically to send fresh sensing fluid from reservoir 118 into sensing zone 110.

In some embodiments, the shape of the fluid channel in the sensing zone may affect the ability of fresh sensing fluid to completely displace used sensing fluid within the sensing zone during calibration. In addition, movement of the fresh sensing fluid may not completely entrain existing sensing fluid in the multiple fluid paths through the tissue piercing elements. It is therefore possible that residual concentration of analyte or interfering species may remain within the sensing zone and may affect the accuracy of the calibration or the accuracy of the analyte concentration determination. For these reasons, the device and method of this invention provide a dwell time for the fresh sensing fluid. During the dwell time, a bolus of fresh sensing fluid remains stationary or substantially stationary within the sensing zone so that residual analyte or interfering species can diffuse or otherwise migrate from hard to reach portions of the sensing zone. At the end of the dwell time, this first bolus of sensing fluid is displaced by a second bolus of fresh sensing fluid. Further boluses may be provided to reduce the concentration of the analyte or interfering species to an acceptable level.

In addition, even apart from calibration, it may be desirable to periodically remove interfering species from the sensing zone. Undesirable species may refer to interfering species that react on the sensor electrode to cause extraneous non-analyte-related signal. Interfering species may be endogenous or exogenous compounds affecting the response of the analyte sensor. Such species can interfere with the proper functioning of the sensor in several ways. First, they can oxidize on (or otherwise react with) the sensor electrode, thereby altering the sensor signal in a manner not related to the analyte of interest. Ascorbic acid, uric acid, and acetaminophen are three examples of compounds that interfere in this way. Even if the sensor itself has an anti-interference membrane, the concentration of the interferents will increase in the sensing fluid and eventually become so high that it could overwhelm the ability of the membrane to exclude the interferents.

Second, proteins and other large biomolecules can adsorb onto the surface of the sensor (either the electrodes or the chemistry layer) and create a diffusion barrier to glucose. This hindered diffusion will manifest itself in a longer lag time for the sensor.

Third, species can build up in the sensing fluid that react with the H₂O₂ produced by the glucose-glucose oxidase reaction before the H₂O₂ can diffuse to the electrode to be detected. This H₂O₂ depletion will cause a steadily decreasing sensitivity of the system to glucose as the species increase in concentration over time. (Because not all of the H₂O₂ created will be consumed by the H₂O₂-depleting species, it is expected that concentration will increase over time if the flux of the H₂O₂-depleting species is similar to that of glucose.) A steadily decreasing sensitivity will eventually limit the operating lifetime of the system as the S/N decreases to the point where the accuracy of the measurement is compromised past the point where it can be corrected for by algorithms, etc.

Undesirable species can also change the hydrophilicity of a surface of the sensor, which can adversely affect sensor operation.

Periodically flushing the sensor chamber with a solution will remove the interfering species that have diffused into the chamber. Flushing may also remove some species that have adsorbed onto the sensor, if the adsorption is reversible. The flush fluid could be the same fluid as the calibration fluid, that is, it could contain a known concentration of glucose. If the flush fluid is the calibration fluid, then the flush period could be increased by a time sufficient to flush the sensor volume before the calibration cycle begins. Alternately, a separate flush fluid could be used which contains no glucose. It could comprise other components, however, including buffer salts, electrolyte salts, surfactants, etc. It could also comprise components specifically known to assist in removing the accumulated interfering species, for example, by removing adsorbed proteins.

In some embodiments, microneedle array 102, reservoirs 118 and 126, channel 108, sensor 112 and adhesive pads 116 are contained within a support structure (such as a housing 128) separate from electronics element 114 and actuator 120, which are supported within their own housing 130. This arrangement permits the sensor, sensing fluid and tissue piercing elements or fluid paths to be discarded after a period of use (e.g., when reservoir 118 is depleted) while enabling the electronics and actuator to be reused. A flexible covering (made, e.g., of polyester or other plastic-like material) may surround and support the disposable components. In particular, the interface between actuator 120 and reservoir 118 must permit actuator 120 to move sensing fluid out of reservoir 118, such as by deforming a wall of the reservoir. In these embodiments, housings 128 and 130 may have a mechanical connection, such as a snap or interference fit.

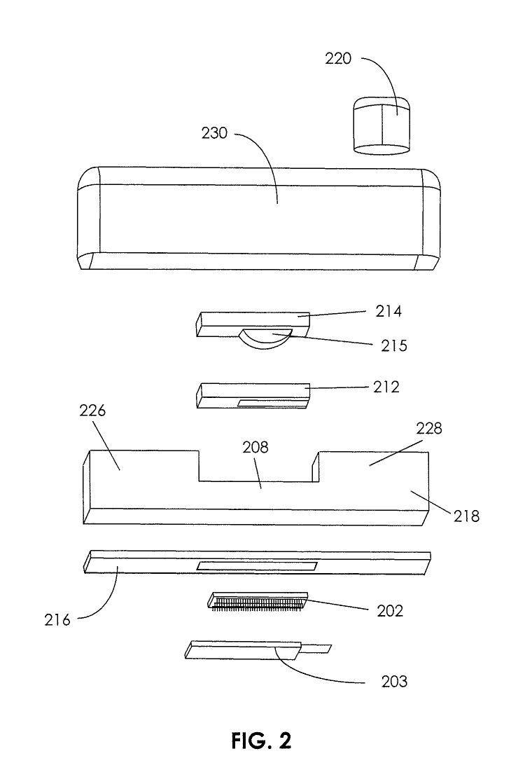

FIG. 2 shows an exploded view of another embodiment of the invention. This figure shows a removable seal 203 covering the sharp distal ends of tissue piercing elements or fluid paths 202 and attached, e.g., by adhesive. Seal 203 maintains the sensing fluid within the tissue piercing elements or fluid paths and sensing zone prior to use and is removed prior to placing the glucose monitor 200 on the skin using adhesive pressure seal 216. In this embodiment, tissue piercing elements or fluid paths 202, sensing fluid and waste reservoirs 218 and 226, sensing microchannel 208 and electrochemical glucose sensor 212 are contained within and/or supported by a housing 228 which forms the disposable portion of the device. A second housing 230 supports an electronics board 214 (containing, e.g., processing circuitry, a power source, transmission circuitry, etc.) and an actuator 220 that can be used to move sensing fluid out of reservoir 218, through microchannel 208 into waste reservoir 226. Electrical contacts 215 extend from electronics board 214 to make contact with corresponding electrodes in glucose sensor 212 when the device is assembled.

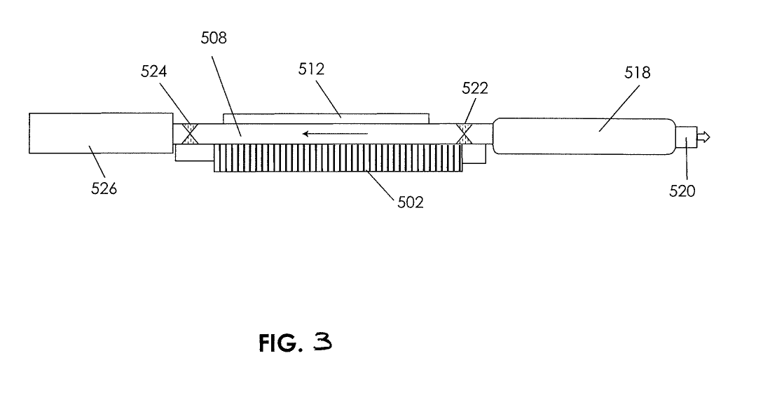

Another embodiment of the disposable portion of the glucose monitor invention is shown in FIG. 3 with a microneedle array 502 and a glucose sensor 512 in fluid communication with a sensing zone in channel 508. In this embodiment, actuator 520 (e.g., a pulsatile pump) is on the side of sensing fluid reservoir 518, and the waste reservoir 526 is expandable. Operation of actuator 520 sends sensing fluid from reservoir 518 through one way flap valve 522 into the sensing zone in channel 508 and forces sensing fluid within channel 508 through flap valve 524 into the expandable waste reservoir 526.

In the embodiment of FIG. 3 (and potentially other embodiments), the starting amount of sensing fluid in the calibration reservoir 518 is about 1.0 ml or less, and operation of the sensing fluid actuator 520 sends a few microliters (e.g., 10 μL) of sensing fluid into channel 508. Recalibrating the device three times a day for seven days will use less than 250 μL of sensing fluid.

As in the other embodiments, the shape of the fluid channel in the sensing zone may affect the ability of fresh sensing fluid to completely displace used sensing fluid within the sensing zone. In addition, movement of the fresh sensing fluid may not completely entrain sensing fluid in the multiple fluid paths through the tissue piercing elements. It is therefore possible that residual concentration of analyte or interfering species may remain within the sensing zone and may affect the accuracy of the calibration or the accuracy of the analyte concentration determination. For these reasons, the device and method of this invention provide a dwell time for the fresh sensing fluid. During the dwell time, a bolus of fresh sensing fluid remains stationary or substantially stationary within the sensing zone so that residual analyte or interfering species can diffuse or otherwise migrate from hard to reach portions of the sensing zone. At the end of the dwell time, this first bolus of sensing fluid is displaced by a second bolus of fresh sensing fluid. Further boluses may be provided to reduce the concentration of the analyte or interfering species to an acceptable level.

A variety of pumps and other fluid acutators may be used with this invention. In one embodiment, the pump is a pulsatile pump. For example, the pump may include a mechanical push down membrane, a membrane attached to a motor, a shape memory alloy or electro-mechanical arrangement. The pump may be configured to operate with a dwell time between pump strokes. The dwell time may range from 1 to 60 seconds, preferably 1 to 30 seconds. The pump may be configured to be operated manually, or it may be programmed to operate automatically in a manner controlled by a microprocessor or other controller.

The dwell time may be based on changing of a duty cycle of the pump. The relationship between a pumping duty cycle and the efficiency of flushing a sensing chamber of fixed volume may be related to the flow rate of the pump, the cell geometry, and the consumption rate of the analyte by the sensor enzyme.

The dwell time may also be based on varying a rotational speed of a motor in the sensing fluid reservoir. The pulsatile pump may be configured to operate based on a speed of an actuator in the pump. The pulsatile pump may also be configured to operate based on a profile of an actuator in the pump.

In particular, if a pumping rate is varied by varying the dwell time between individual pump actuations, there is a dwell time where the flushing efficiency is optimized. This optimum is related to the geometry of the sensor cell and the consumption rate of the sensor. With too short a dwell time (fast pumping), a large volume of liquid will be passed through the cell without efficiently flushing the volume around the walls of the cell. With long dwell times (slow pumping), a significant fraction of the glucose is delivered into the cell and reacts with the glucose oxidase to form H₂O₂ which either degrades passively or is swept out of the cell by subsequent pump pulses.

In some embodiments described above, sensing fluid within the sensing zone is flushed and displaced by new sensing fluid, in other words, the flushing fluid is sensing fluid, i.e., it has the same components and component concentrations as the sensing fluid. In other embodiments, the flushing fluid may be different than the sensing fluid, e.g., with different components and component concentrations. For example, the flushing fluid may be calibration fluid with a glucose concentration higher or lower than the glucose concentration of fresh sensing fluid. The flushing fluid may also have zero concentration of glucose (or other analyte) or an interfering species thereof in order to maximize the rate of diffusion of lingering analyte or species during the dwell time following displacement of the sensing fluid in flushing operation.

The flushing schedule can be chosen to meet the needs of a particular sensor. For example, the sensing chamber may be flushed every two hours or every four hours for a continuous glucose monitor and not every time the glucose sensing is performed.

While exemplary embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of monitoring an analyte comprising:
diffusing the analyte from a sampling location into a sensing fluid within a sensing chamber;
detecting a concentration of the analyte in the sensing fluid;
moving flushing fluid into the sensing chamber and simultaneously removing sensing fluid from the sensing chamber;
permitting the flushing fluid to remain in the sensing chamber without flowing for a dwell time;
removing the flushing fluid from the sensing chamber after the dwell time expires; and
after removing the flushing fluid from the sensing chamber, moving sensing fluid into the sensing chamber and detecting a concentration of the analyte in the sensing fluid.

2. The method of claim 1 wherein the dwell time is between 1 second and 30 seconds.

3. The method of claim 1 wherein the steps of moving and removing the flushing fluid are performed by a pulsatile pump.

4. The method of claim 1 wherein the analyte is glucose.

5. The method of claim 1 wherein the sampling location is a subject's interstitial fluid.

6. The method of claim 5 wherein diffusing comprises diffusing the analyte from the interstitial fluid via fluid paths formed through a stratum corneum layer of the subject's skin.

7. The method of claim 6 further comprising forming the fluid paths through the stratum corneum.

8. The method of claim 1 wherein the flushing fluid and the sensing fluid are the same.

9. The method of claim 1 wherein the diffusing step comprises diffusing the analyte from the sampling location into the sensing fluid without using a dialysis membrane.

10. The method of claim 1 wherein the flushing fluid is an aqueous buffer electrolyte solution.

11. The method of claim 1 wherein the flushing fluid is calibration fluid, the method further comprising calibrating an analyte sensor prior to removing the flushing fluid from the sensing chamber.

12. An analyte monitoring device comprising:
a sampling member;
a sensing chamber in fluid communication with the sampling member;
an analyte sensor in fluid contact with the sensing chamber; and
a pump programmed to move flushing fluid into the sensing chamber and remove sensing fluid from the sensing chamber so that the flushing fluid remains in the sensing chamber without flowing for a dwell time.

13. The device of claim 12 wherein the sampling member comprises a fluid path.

14. The device of claim 12 wherein the sampling member comprises a tissue piercing element.

15. The device of claim 12 wherein the pump comprises a pulsatile pump.
